(19) United States
(12) Patent Application Publication
(10) Pub. No.: US 2008/0071242 A1
(43) Pub. Date: Mar. 20, 2008

(54) IRRIGATION BAG WITH INHERENT FLUID COMPRESSION MEANS AND INSULATION

(76) Inventor: George C. Christoudias, Saddle River, NJ (US)

Correspondence Address: RICHARD A. JOEL ESQ., 496 KINDERKAMACK ROAD, ORADELL, NJ, 07649, US

(21) Appl. No.: 11/422,806

(22) Filed: Jun. 7, 2006

(51) Int. Cl. A61B 19/00

(52) U.S. Cl. 604/408

(57) ABSTRACT

An irrigation/infusion bag comprises a central bag containing irrigation/infusion fluid connected to the tubing delivery system. A peripheral bag surrounds the central bag and includes an insufflation system. When inflated with a hand pump, the peripheral bag compresses the central bag to help deliver the fluid from the irrigation system under pressure and at a desired temperature maintained by the peripheral insulation around the central bag.

An irrigation bag comprises a central generally plastic bag containing irrigation fluid having an outlet connected to the tubing delivery system. The aforesaid bag is surrounded by a peripheral bag coupled to an insufflation system. When the peripheral bag is inflated, it compresses the central bag to deliver the irrigation fluid under pressure and at body temperature due to the insulation qualities of the peripheral bag.

The prior art discloses a flexible irrigation bag having an upper and lower surface and a body portion joining said surfaces to hold fluid. Any pressure on the bag sides would force it to balloon upwardly and/or downwardly defeating the purpose of providing a predetermined irrigation stream.

A typical infusible pressure or intravenous bag is sold by Vital Signs, Inc., under the trademark Infusible®. The device includes a sealed sleeve to hold a bag of irrigation fluid, a bottom outlet and a hose for pumping air into the sleeve to force fluid outwardly from the outlet. The device is described in detail in U.S. Pat. No. 4,735,613 to Bellin et al.

Another prior art device is U.S. Pat. No. 4,090,514 to Hinck, et al. The bag for applying pressure to the sealed liquid bag is of a permanent nature and must be cleaned and sanitized after each use.

Other prior art patents of interest include U.S. Pat. No. 6,419,662 to Solazzo on a continuous irrigation Y-tubing control valve device and system and U.S. Pat. No. 6,685,667 to Delk, et al on an electronically powered surgical irrigator.

Patent Publication US 2004/0097872 discloses a surgical irrigation system particularly well suited for laparoscopic surgery. The system includes a reusable console and a disposable pump unit with a pump/motor module, a hand-piece and tubing, which connects the pump/motor module to an irrigation bag.

Finally, Patent Publication US 2005/0025646 discloses a foot pedal irrigation system.

The present invention is an improvement over the prior art which is inexpensive, effective and easy to use. The compression means is hand operated eliminating the need for a motor and foot pump.

The present invention comprises a central bag containing irrigation/infusion fluid, which is connected to the tubing system for gravity flow. An insulated outer bag surrounds the central bag and is connected to a hand operated insufflation system. Inflating the outer bag with a pump in the hand operated insufflation system increases the flow of irrigation fluid from the central bag. The irrigation fluid is heated to the desired temperature and then maintained to that temperature by the insulation on the outer bag and the hand operated pump eliminates the need for a noisy foot-operated floor pump.

Accordingly, it is an object of this invention to provide a new and improved irrigation bag.

Another object of this invention is to provide a new and improved irrigation bag with inherent fluid compression means to provide flow at a predetermined rate into an irrigation tubing system.

A further object of this invention is to provide a new and improved irrigation bag, which is efficient, easy to use and inexpensive.

A still further object of this invention is to provide an irrigation bag, which includes a hand pump coupled to an external bag for uniformly compressing irrigation fluid and/or blood in an internal bag and maintaining the temperature of the fluid.

The above and other objects and advantages of the present invention may be more clearly seen when viewed in conjunction with the accompanying drawings wherein:

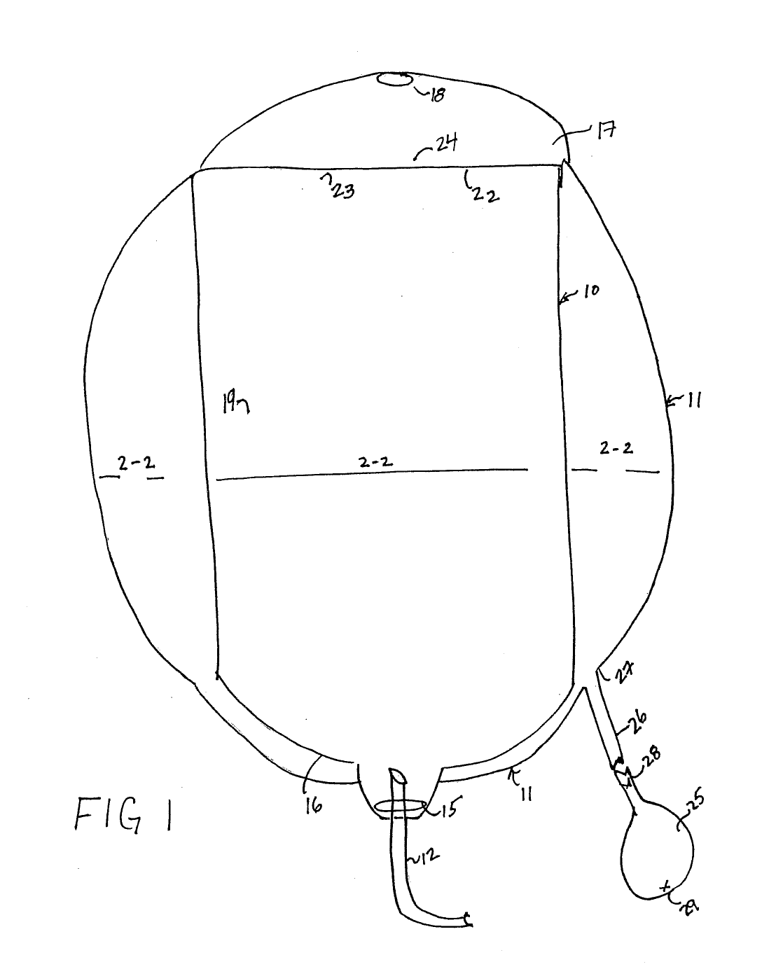

FIG. 1 is a schematic view of the irrigation bag proposed by this invention.

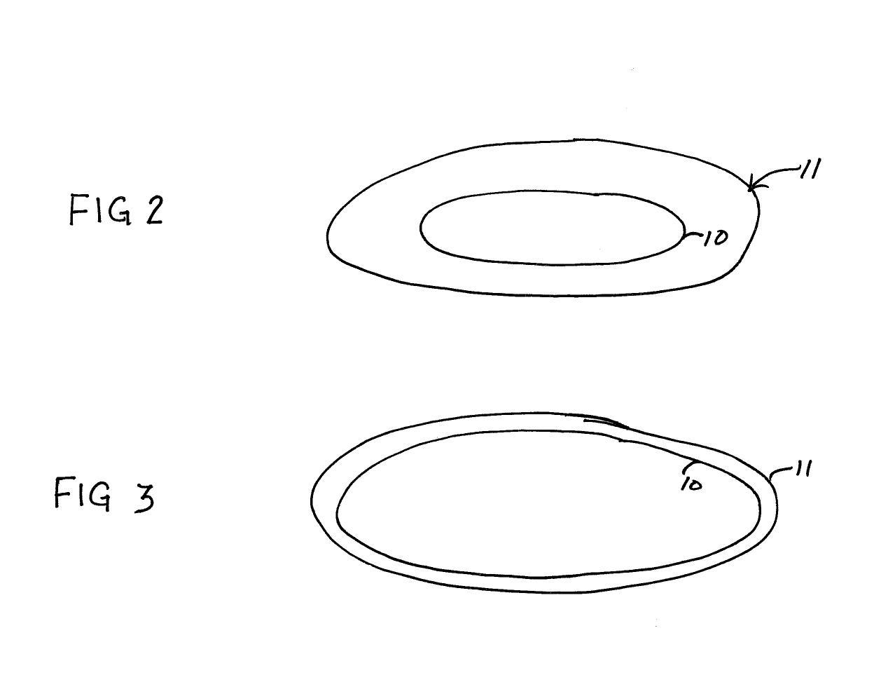

FIG. 2 is a cross-section of the irrigation bag taken along the line 2-2 of FIG. 1 with the peripheral bag inflated; and,

FIG. 3 is a cross-section of the irrigation bag taken along the line 2-2 of FIG. 1 with the peripheral bag not inflated.

This invention relates to an irrigation bag 10 which supplies irrigation fluid and/or blood through an outlet 13 to a tubing 12 connected to the irrigation system. The tubing 12 includes a coupling 15, which secures the tubing 12 to the outlet 13 in the bottom 16 of the irrigation bag 10. The bag 10 also includes a flat top portion 17 including an aperture 18 for connecting to a supporting hook (not shown).

The central irrigation bag 10 is completely surrounded by a peripheral bag 11 on its elongated sides 19 and bottom 16. The irrigation bag 10 and the peripheral bag 11 are sealed along the line of connection 22 on the upper portions 23 and 24. A hand operated insufflation device 25 is connected by tubing 26 to an aperture 27 in the flexible balloon peripheral bag 11. Check valves 28 and 29 permit a one-way flow of air when the flexible device 25 is squeezed inflating peripheral bag 14. This eliminates the need for a noisy foot operated motorized pump, which is difficult to control.

FIG. 2 depicts the central irrigation bag 10 with the peripheral bag 11 inflated forcing the irrigation fluid outwardly from the aperture 13. On the other hand, FIG. 3 depicts the central bag 10 filled with fluid and depicts the peripheral bag 11 in a not inflated condition.

In operation, the surrounding peripheral bag 11 is insufflated with the hand pump 25. The bag 11 surrounds and squeezes the central bag 10 forcing irrigation fluid through the outlet tubing 12 to the desired location. This eliminates the need for a floor pump and motor; and provides more precise control of the flow of irrigation fluid.

The flexible inflatable device or balloon 25 forces air which enters through valve 29 through the check valve 28 and tubing 26 into bag 14. The bag 11 inflates under pressure and forces the sides 19 and 21 inwardly expelling irrigation fluid through the bottom outlet 13. The doctor operating on a patient does not have to search for a floor pedal particularly if the operating is dark.

The irrigation arrangement described herein is inexpensive, easy to use and provides superior control over the flow of fluid. Since the peripheral bag 11 with its insulating qualities surrounds bag 10, the irrigation fluid is maintained at a preheated and desired temperature usually body temperature, which is appropriate for irrigation purposes and delivers the fluid under appropriate pressure.

While the invention has been explained by a detailed description of certain specific embodiments, it is understood that various modifications and substitutions can be made in any of them within the scope of the appended claims that are intended also to include equivalents of such embodiments.

What is claimed is:

1. An irrigation/infusion bag for dispensing irrigated or infused fluid, blood or blood products for medical use to a selected site comprises: a central flexible plastic bag for holding irrigation/infusion fluid, blood or blood products having an upper portion, a side portion and a lower portion, said lower portion having an outlet aperture; an outer inflatable peripheral bag surrounding the sides and bottom of the central plastic bag and being connected to the upper portion thereof, said inflatable bag having an air inlet; an insufflation pump connected to the air inlet of the outer bag for inflating the outer inflatable bag to force fluid from the outlet aperture of the central flexible bag; and, tubing connecting the outlet aperture to the irrigation site.

2. An irrigation/infusion bag in accordance with claim 1 wherein: the outer peripheral bag is insulated by the instilled air to maintain the fluid in the central bag at a pre-heated body temperature.

3. An irrigation/infusion bag in accordance with claim 1 wherein: the insufflation pump comprises a flexible hollow balloon, which may be squeezed to force air outwardly therefrom into the peripheral bag to exert a predetermined pressure on the central bag to force a predetermined fluid flow outwardly therefrom.

4. An irrigation/infusion bag in accordance with claim 3 wherein: the insufflation pump includes tubing connected to its outlet at one end and to the air inlet of the outer bag at its other end.

5. An irrigation/infusion bag in accordance with claim 4 wherein: the tubing includes a check valve mounted in the tubing between the pump and the peripheral bag.

6. An irrigation bag/infusion in accordance with claim 1 wherein: the connection to the upper portion of the central bag to the outer peripheral bag is sealed to the upper portion of the central bag.

7. An irrigation bag/infusion in accordance with claim 1 wherein: the insufflation pump will insufflate the outer bag to a degree of pressure sufficient to deliver the irrigation fluid.

8. An irrigation bag/infusion in accordance with claim 7 further including: a portion extending upwardly from the irrigation bag having a holding aperture therein.
